(19) United States
(12) Patent Application Publication
(10) Pub. No.: US 2004/0187869 A1
(43) Pub. Date: Sep. 30, 2004

(54) Training device for medicament inhalers

(75) Inventors: Paul M. Bjorndal, Wayne, NJ (US); Jun Chen, Warren, NJ (US); David J. Kenyon, Morristown, NJ (US); Barry N. Lutsky, Hillsborough, NJ (US); Keith B. Nolop, Redwood City, CA (US); John M. Stimson, New Providence, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/759,859

(22) Filed: Jan. 16, 2004

Related U.S. Application Data
(60) Provisional application No. 60/440,831, filed on Jan. 17, 2003.

(51) Int. Cl. A61M 15/00; A61M 16/00

(52) U.S. Cl. 128/203.15; 128/200.23; 128/203.12

(57) ABSTRACT

A training device for a dry powder inhaler, includes a dry powder inhaler simulator including a housing having a bore extending therethrough, a mouthpiece connected with the housing and being in open communication with the bore; a control circuit for measuring pressure drop at the opening and displaying a single value corresponding to both inhalation rapidity and inhalation flow rate peak, the control circuit including a pressure transducer below the opening for producing an output signal corresponding to the pressure drop, an A/D converter for converting the output signal to a digitized signal; a display formed by a plurality of lighting segments, for providing an indication corresponding to both inhalation rapidity and inhalation flow rate peak, and a microprocessor connected with the display for controlling the display in response to the output signal from the pressure transducer and elapsed time.

The present invention relates generally to medication dispenser assemblies used for inhalation of a measured dose of a medicament and, more particularly, is directed to a device for training a patient to use such a medication dispenser assembly to achieve an optimized result.

When delivering inhalation medicaments, that is, pharmacologically active compounds, in solid form to the respiratory tract and to the lungs, careful attention to the accuracy of the dosage, which can be smaller than 0.1 milligram, must be made. This is because such medicaments are often quite potent, and the administration of excessive amounts thereof could be harmful to the patient. Further, if the dosage that is delivered is too small, it will not serve its purpose.

It is also necessary that the medicament particles leaving the dispenser assembly be substantially within a particular size range, since particles of the medicament which are too large may not enter a desired lower portion of the respiratory tract, such as the bronchial tree or lungs, but instead will be deposited in the mouth or pharynx and thence enter the digestive tract. As an example, preferred particles for inhalation into the lower airway usually are considered as having a diameter less than about 10 micrometers.

Various devices have been used in order to dispense a metered dose of powdered medicament, including pressurized aerosol devices, insufflation devices, pump inhalators and the like. With the current concern over environmental issues, however, pressurized aerosol devices, which constitute a large part of the devices now on the market, are less favored.

In addition to the aforementioned types of dispenser assemblies, dry powder inhalers are also known. Studies have shown that there are typically no significant differences in bronchodilator responses with equivalent amounts of medicinal substances administered either by dry powder inhalers or aerosol devices. Accordingly, there is now an ever-growing demand for dry powder inhalers which can dispense measured doses of powdered medicament. With such devices, the powder is automatically withdrawn during inspiration so there is less need to be concerned with synchronizing release of medication with the exact start of inspiration to insure quality of the product delivery.

Examples of multiple dose dry powder inhalers are described in Published International Patent Application Numbers: WO 93/00123; WO 94/14492; and WO 97/30743; as well as in U.S. Pat. Nos. 5,394,868; 5,687,710; 5,740,792; 5,829,434; and 6,240,918; and the entire disclosures of these documents are incorporated herein by reference. A dry powder inhaler in accordance with the teachings of these patents will be sold by Schering-Plough Corporation and its affiliates under the trademark “TWISTHALER”. Other examples of dry powder inhalers include those being sold by other companies under the trademarks “TURBUHALER,” “ACCUHALER” and “DISKHALER.”

Dry powder inhalers differ from the classical propellant-based metered dose pressurized aerosol inhalers in which there is a coordinated action of depressing the canister into an actuator housing to actuate a metering valve, and inhalation. Rather, with dry powder inhalers, it is the action of inhalation by itself which initiates and powers the delivery of a measured dose of dry powder to the patient's respiratory tract.

However, because the patient's inhalation provides the sole power for the dry powder inhaler, the inhalation velocity must be greater than a critical minimum value in order to produce respirable particles exiting the dry power inhaler, since the drug supply within the inhaler can comprise powder particle agglomerates which are too large for inhalation and these must be de-agglomerated by means of airflow path changes and other internal features of the inhaler, generally functioning properly only if the airflow velocity exceeds the particular minimum value. The effect of the therapy is reduced if the user cannot attain the critical inhalation minimum velocity or flow within a given time, and the patient may not receive the desired amount of medication.

Thus, optimum use of the dry powder inhaler can require a certain degree of patient education and training. Proper dry powder inhaler usage requires the patient to take a relatively rapid, deep breath in order to generate adequate flow through the inhalation channel of the dry powder inhaler. This differs from usage of a pressurized aerosol metered dose inhaler which requires the patient to take a slow, steady breath.

In general, an inspiratory flow rate of at least 60 Liters per minute (L/min) is considered optimal for efficient dose delivery from a dry powder inhaler. However, patients must generally be able to generate a minimum inspiratory flow rate of about 20 L/min to achieve efficient drug delivery in the dry powder inhaler to be sold under the trademark “ASMANEX TWISTHALER” by Schering-Plough Corporation and its affiliates, which dry powder inhaler is described in the above-noted patents. By contrast, a minimum inspiratory flow rate from the dry powder inhaler sold by AstraZeneca LP under the trademark “PULMICORT TURBUHALER” is approximately 30 L/min.

For this reason, it is desirable to provide a device for training a patient to properly use a particular medicament inhaler to ensure that there will be a rapid enough breath with at least a certain minimum inspiratory flow rate.

### SUMMARY OF THE INVENTION

In accordance with an aspect of the present invention, a training device for a medication inhaler includes a medication inhaler simulator having a housing with a bore extending therethrough, and a mouthpiece connected with the housing and being in open communication with the bore, and a control circuit for measuring pressure drop at a position below the mouthpiece, the control circuit including a pressure transducer for producing an output signal corresponding to the pressure drop at the position, a display for providing an indication of acceptable inhalation, and a microprocessor connected with the display for controlling the display in response to the output signal from the pressure transducer and elapsed time.

In one embodiment, the medication inhaler simulator further includes an opening in a wall of the housing; and a conduit having one end connected with the wall of the housing at the opening and an opposite end connected with the pressure transducer, wherein the control circuit measures the pressure drop at the opening.

In one embodiment, the housing may include a restriction arrangement for restricting air passage through a portion of the bore to provide adjustment of a pressure drop through the bore. The opening in the housing is positioned between the restriction arrangement and the mouthpiece.

In another embodiment, the mouthpiece is removably mounted on the housing, and specifically is threadedly mounted on the housing. In this embodiment, the housing includes a closed end covered by the mouthpiece, the closed end including a pressure sensing opening positioned above the pressure transducer, and at least one air flow opening fluidly connected with an outside of the housing. The housing includes at least one recessed wall section positioned below the at least one air flow opening, and the recessed wall section includes a curvature that reduces in depth in a radial dimension at an intermediate portion thereof to create an air flow restriction with the mouthpiece so as to create a venturi effect.

The control circuit includes an analog-to-digital converter connected between the pressure transducer and the microprocessor for digitizing the output signal prior to supply thereof to the microprocessor.

The control circuit also includes a start switch for closing a power circuit in order to supply power to the microprocessor from a power supply. A timer circuit is also desirably provided for opening the power circuit after a predetermined amount of time.

A voltage regulator is connected with the power supply through the power circuit, and supplies a predetermined DC voltage to the microprocessor in response to power supplied by the power supply.

A voltage detector is connected with the microprocessor, for detecting voltage supplied by the power supply, and for sending a signal to the microprocessor when the voltage is below a predetermined value in order to prevent operation of the microprocessor.

The display includes at least one lighting device which is selectively caused to be illuminated by the microprocessor for providing an indication of both inhalation rapidity and inhalation flow rate peak. Preferably, the at least one lighting device includes a plurality of light emitting diodes, and further includes drivers connected between the light emitting diodes and the microprocessor. Also, the lighting device is preferably arranged to display the inhalation rapidity and the inhalation flow rate peak in a bar graph form. In another preferred embodiment, the at least one lighting device includes a liquid crystal display.

In another embodiment, the display includes a lighting device which is selectively caused to be illuminated by the microprocessor to provide a single display which indicates whether inhalation is acceptable. The single display is a function of values of both inhalation rapidity and inhalation flow rate peak, and includes a plurality of different colored bars which are selectively illuminated in dependence upon a single value calculated from the values of both inhalation rapidity and inhalation flow rate peak. Specifically, there are a plurality “n” of the bars which are selectively illuminated in dependence upon the single value calculated as follows: if the values of both inhalation rapidity and inhalation flow rate peak are below a threshold value “m” which is less than n, the lower value of inhalation rapidity and inhalation flow rate peak is displayed as the single value, providing that the flow rate at a predetermined time from the start of inhalation is above a predetermined flow rate, to indicate an unsuccessful inhalation, wherein a number of the bars corresponding to the single value are illuminated. If the values of both inhalation rapidity and inhalation flow rate peak are at least as high as the threshold value, an average truncated value of the values of both inhalation rapidity and inhalation flow rate peak is displayed as the single value, providing that the flow rate at the predetermined time from the start of inhalation is above the predetermined flow rate, to indicate a successful inhalation, wherein a number of the bars corresponding to the single value are illuminated. If the flow rate at the predetermined time from the start of inhalation is below the predetermined flow rate, this indicates an unsuccessful inhalation, and a number of the bars corresponding to the single value are illuminated. Preferably, m=5 and n=10.

In accordance with another embodiment, the control circuit is mounted within the housing. In such case, the housing includes a viewing opening, and the display is positioned to be viewed through the viewing opening.

In one embodiment, the mouthpiece includes a one-way valve arrangement to inhibit contamination of the housing if a patient exhales into the mouthpiece. The one-way valve arrangement includes a retainer having openings mounted in the mouthpiece, and a flexible valve flap mounted on the retainer which moves away from the retainer to permit air flow through the retainer and the mouthpiece during inhalation and which blocks the openings in the retainer during exhale by the patient through the mouthpiece. Further, the control circuit includes a start switch for closing a power circuit in order to supply power to the microprocessor from a power supply; and the device further includes a closure cap removably positioned over the mouthpiece, and an actuation assembly in the housing for actuating the start switch upon removal of the closure cap. The actuation assembly includes a ring mounted for rotation in the housing, the ring including an actuating projection for actuating the start switch upon rotation of the ring, and a first engaging portion; and a second engaging portion mounted to the closure cap for engagement with the first engaging portion to rotate the ring. Specifically, the housing includes external threads, and the closure cap includes internal threads for threadedly positioning the closure cap on the housing in covering relation to the mouthpiece; the first engaging portion extends outwardly of the housing through at least one opening in a wall of the housing; and the second engaging portion is formed on an inner surface of the closure cap so as to rotate the ring in a first direction upon threaded removal of the closure cap and to rotate the ring in a second direction upon threaded insertion of the closure cap on the housing.

In accordance with another aspect of the present invention, a method of assessing a patient's inhalation process with respect to a medication inhaler includes the steps of inhaling through a mouthpiece of an inhaler simulator having a housing with a bore extending therethrough, with the mouthpiece connected with the housing and being in open communication with the bore; measuring a pressure drop at a position below the mouthpiece during inhalation through the mouthpiece, and providing an output signal in response thereto; determining inhalation rapidity in response to the output signal and an elapsed time; determining an inhalation flow rate peak in response to the output signal; and providing a display in response to both the inhalation rapidity and the inhalation flow rate peak. The steps of determining inhalation rapidity and determining inhalation flow rate peak are performed in a microprocessor, and the output signal is digitized prior to supply thereof to the microprocessor.

Also, power to the microprocessor is preferably shut off after a predetermined amount of time.

The step of determining inhalation rapidity includes the steps of: reading the output signal; determining a starting time when the output signal reaches a predetermined initial value; determining an ending time when the output signal reaches a predetermined ending value; determining a time period between the initial value and the ending value; and determining the inhalation rapidity from a difference between the ending value and the initial value over the time period.

The step of displaying includes the step of illuminating at least one lighting device to display the inhalation rapidity and the inhalation flow rate peak in a bar graph form.

In a preferred embodiment, the step of displaying includes the step of illuminating at least one lighting device to display an indication of whether inhalation is acceptable. Specifically, a plurality of bars are selectively illuminated in dependence upon a single value calculated from the values of both inhalation rapidity and inhalation flow rate peak. Preferably, different ones of the display bars are illuminated with different colors. In this case, there are a plurality n of the bars, and the step of displaying includes the following steps. At least one bar corresponding to a single lower value of inhalation rapidity and inhalation flow rate peak is illuminated if the values of both inhalation rapidity and inhalation flow rate peak are below a threshold value m which is less than n, providing that the flow rate at a predetermined time from the start of inhalation is above a predetermined flow rate, to indicate an unsuccessful inhalation. A number of the bars corresponding to an average truncated value of the values of both inhalation rapidity and inhalation flow rate peak is illuminated if the values of both inhalation rapidity and inhalation flow rate peak are at least as high as the threshold value, providing that the flow rate at the predetermined time from the start of inhalation is above the predetermined flow rate, to indicate a successful inhalation. At least one bar corresponding to a single value is illuminated if the flow rate at the predetermined time from the start of inhalation is below the predetermined flow rate, to indicate an unsuccessful inhalation. In a preferred embodiment, m=5 and n=10.

The above and other objects, features and advantages of the invention will become readily apparent from the following detailed description thereof which is to be read in connection with the accompanying drawings.

### BRIEF DESCRIPTION OF THE DRAWINGS

FIG. 1 is a plan view of a training device for a dry powder inhaler according to the present invention;

FIG. 2 is a circuit wiring-block diagram of the circuitry of the training device of FIG. 1;

FIG. 3 is a plan view of an alternative display for the training device of FIG. 1;

FIG. 4 is a graph of inspiratory flow rate versus time for adults with the training device of FIG. 1;

FIG. 5 is a graph of inspiratory flow rate versus time for children with the training device of FIG. 1;

FIG. 6 is a lengthwise cross-sectional view of a training device according to another embodiment of the present invention, showing arrangement of internal components therein;

FIG. 7 is a side elevational view of a lower portion of the housing of FIG. 6;

FIG. 8 is a cross-sectional view of the housing of FIG. 7, taken along line 8-8 thereof;

FIG. 9 is a perspective view of a removable mouthpiece for use with the training device of FIG. 6;

FIG. 10 is a top plan view of the mouthpiece of FIG. 9;

FIG. 11 is a cross-sectional view of the mouthpiece of FIG. 9, taken along line 11-11 of FIG. 10;

FIG. 12 is a cross-sectional view of the mouthpiece of FIG. 9, taken along line 12-12 of FIG. 10;

FIG. 13 is a bottom plan view of the mouthpiece of FIG. 9;

FIG. 14 is a side elevational view of the mouthpiece of FIG. 9;

FIG. 15 is a perspective view of a closure cap for use with the training device of FIG. 6;

FIG. 16 is a cross-sectional view of the closure cap of FIG. 15, taken along line 16-16 thereof;

FIG. 17 is a cross-sectional view of the closure cap of FIG. 15, taken along line 17-17 thereof;

FIG. 18 is a front perspective view of the housing of a training device according to still another embodiment of the present invention;

FIG. 19 is a rear perspective view of the housing of FIG. 18;

FIG. 20 is a rear elevational view of the housing of FIG. 18;

FIG. 21 is a side elevational view of a portion of the housing of FIG. 18;

FIG. 22 is a top plan view of the housing of FIG. 18;

FIG. 23 is a perspective view, partially broken away of the mouthpiece assembly for use with the housing of FIG. 18;

FIG. 24 is a perspective view of a valve flap for use with the mouthpiece assembly of FIG. 23;

FIG. 25 is a perspective view of a retainer for securing the valve flap in the mouthpiece assembly;

FIG. 26 is a perspective view of the planar circuit board mounted in the housing of FIG. 18;

FIG. 27 is a perspective view of a sealing gasket for sealing the pressure transducer on the circuit board when the latter is mounted in the housing;

FIG. 28 is a perspective view of the transparent curved lens to be mounted in the opening in the housing;

FIG. 29 is a perspective view of the lower end cap which closes the lower end of the housing;

FIG. 30 is a circuit wiring diagram of the central processing unit (CPU) or microprocessor, the momentary start button, the battery and the crystal mounted on the circuit board of FIG. 26;

FIG. 31 is a circuit wiring diagram of the pressure transducer circuit which is mounted on the circuit board of FIG. 26;

FIG. 32 is a circuit wiring diagram of the LCD display mounted on the circuit board of FIG. 26; and

FIG. 33 is a bar graph display that is illuminated on the LCD display of FIG. 33, in response to inhalation by a user.

### DETAILED DESCRIPTION

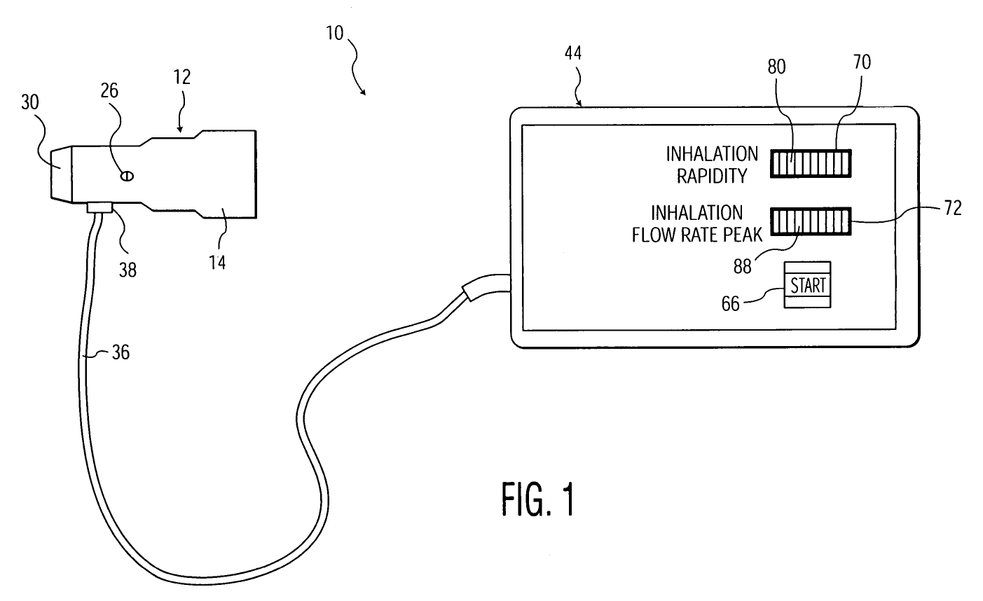

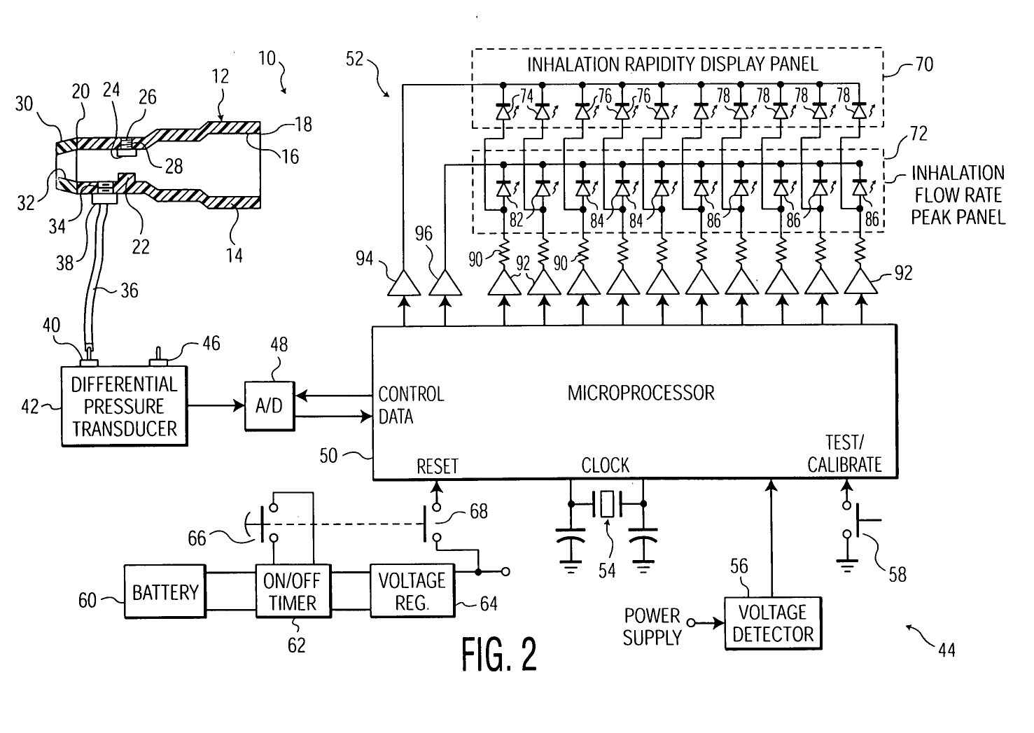

Referring to the drawings in detail, and initially to FIGS. 1 and 2 thereof, a training device 10 according to the present invention, for training a patient to use a dry powder inhaler, includes a dry powder inhaler (DPI) simulator 12. Simulator 12 can take any size and configuration, but is preferably of the same size and configuration as the dry powder inhaler to be used by the patient for actually dispensing the powdered medicament. DPI simulator 12 in FIGS. 1 and 2 is shown with a size and configuration of the dry powder inhaler intended to be sold under the trademark “TWISTHALER,” although the present invention is not so limited.

Simulator 12 includes a housing 14 which is hollow, and thereby includes a central axial bore 16 therein. Bore 16 is shown to increase in diameter toward the lower end, in accordance with the outer configuration of housing 14. This is provided so that the walls of housing 14 are of a uniform thickness. However, the present invention is not limited thereto, and central bore 16 can have a constant thickness throughout, with the walls of housing 14 varying in thickness, or any other suitable arrangement. The lower end 18 and upper end 20 of housing 14 are both open, so that bore 16 constitutes a through bore extending entirely through housing 14.

An annular constriction 22 is provided on the inner wall of housing 14 to provide a reduced bore opening 24 thereat. A set screw 26 is threadedly received in a threaded bore 28 extending radially through housing 14 and constriction 22, and can be turned so as to extend into reduced bore opening 24 to further reduce the opening area thereat, in order to provide a greater pressure drop, or resistance to air flow.

DPI simulator 12 further includes a removable mouthpiece 30 having a central axial bore 32. Mouthpiece 30 can be detachably mounted on upper end 20 of housing 14. The detachable mounting can be accomplished in any suitable manner, such as described in the aforementioned patents. A simple arrangement is the use of a plurality of pins (not shown) at the upper edge of housing 14 at upper end 20 which fit into holes (not shown) in the lower edge of mouthpiece 30.

A radial opening 34 is provided in the wall of housing 14 at a position between reduced bore opening 24 and mouthpiece 30, and one end of a single tube, small diameter, flexible pneumatic hose 36 is connected therein by a threaded coupling member 38 at the end of flexible hose 36. In this regard, as a patient inhales through mouthpiece 30, air is sucked through bore 16, creating a pressure drop at radial opening 34. It will be appreciated that set screw 26 can be used to adjust this pressure drop, and thereby calibrate DPI simulator 12 to accurately simulate the pressure drop in a dry powder inhaler.

The opposite end of flexible hose 36 is connected to a vacuum port 40 of a differential pressure transducer 42 of a control circuit 44. Differential pressure transducer 42 also includes a pressure port 46 vented to atmosphere. Differential pressure transducer 42 is a conventional pressure transducer, for example, as described in detail by Donald G. Fink and Donald Christiansen, Electronics Engineers' Handbook, 3rd Edition, McGraw-Hill Book Company, New York, 1989, at pages 10-25 through 10-30.

Differential pressure transducer 42 provides an analog linearized output signal corresponding to the pressure drop. Preferably, this output signal is adjusted to compensate for temperature variations, and in accordance therewith, a conventional temperature compensation circuit (not shown) is provided therein.

As shown in FIG. 2, the linearized output signal from differential pressure transducer 42 is supplied to an analog-to-digital (A/D) converter 48 which converts the analog output signal to a digital or binary signal, and supplies the digital signal as a digitized data signal to a DATA input of single chip microprocessor 50 that controls a display 52 in accordance with the data from the data signal, so as to display both inhalation flow rate peak and inhalation rapidity. Microprocessor 50 sends a control signal from a CONTROL output thereof to A/D converter 48 to control the same.

The internal clock signal for microprocessor 50 is supplied by a clock crystal 54 to a CLOCK input of microprocessor 50. Further, a voltage detector 56 is connected with an input of microprocessor 50 in order to detect the power supplied by a power supply, and provides a signal to microprocessor 50 to prevent operation thereof if the voltage is below a predetermined value, for example, less than 5 volts.

In addition, a hidden test/calibrate switch 58 is connected with a TEST/CALIBRATE input of microprocessor 50 in order to calibrate microprocessor 50 and to cause microprocessor 50 to perform an internal test to ensure that it is operating properly.

Power to the circuit is supplied by a battery power supply 60 via an ON/OFF timer circuit 62 and a voltage regulator 64. Battery power is used so that training device 10 is portable and easy to hold. In this regard, a momentary start switch or button 66 is connected with ON/OFF timer circuit 62 and is ganged with a switch 68 connected between the output of voltage regulator 64 and a reset input RESET of microprocessor 50.

When start switch 66 is depressed, ON/OFF timer circuit 62 is activated to supply power from battery power supply 60 to voltage regulator 64, and thence to the microprocessor. At the same time, because of the ganged relation of switches 66 and 68, switch 68 is also closed, whereby the voltage supplied by ON/OFF timer circuit 62 is regulated and supplied as a 5 volt regulated voltage supply, through switch 68, to the RESET input of microprocessor 50. ON/OFF timer circuit 62 controls the supply of power to voltage regulator 64 in order to conserve battery power, such that operation is terminated or shut down after a predetermined time period, for example 30 seconds. Thus, after the predetermined time period, ON/OFF timer circuit 62 causes termination of power to microprocessor 50.

Display 52 includes an inhalation rapidity display panel 70 and an inhalation peak flow rate panel 72, both of which may, for example, be configured as three-color bar graph displays. Inhalation rapidity display panel 70 displays a score inversely related to rise time, while inhalation peak flow rate panel 72 displays a flow peak score. The rise time is calculated by measuring the slope or steepness of the inhalation flow rate curve between two fixed pressure readings corresponding to two flow rates, for example, 10 L/min and 30 L/min, at different times, while inhalation flow rate peak is the maximum inhalation flow rate achieved during the entire inhalation process.

As shown in FIG. 2, inhalation rapidity display panel 70 can include two red light emitting diodes (LEDs) 74, three yellow LEDs 76 and five green LEDs 78, each positioned behind a transparent plastic or glass bars 80 in order to light up the respective bar 80. Red LEDs 74 indicate a poor inhalation technique; yellow LEDs 76 indicate a marginal inhalation technique; and green LEDs 78 indicate a good inhalation technique. In like manner, inhalation flow rate peak panel 72 includes two red LEDs 82, three yellow LEDs 84 and five green LEDs 86, each positioned behind a transparent plastic or glass bar 88 in order to light up the respective bar 88. Red LEDs 82 indicate a poor inhalation technique; yellow LEDs 84 indicate a marginal inhalation technique; and green LEDs 86 indicate a good inhalation technique. Of course, the bars can be of any desired color scheme, including having all of the bars showing the same color.

The inputs of LEDs 74-78 and 82-86 are coupled through current limiting resistors 90 to the outputs of ten respective lamp drivers 92, the inputs of which are controlled by microprocessor 50. In addition, microprocessor 50 controls a display multiplex driver 94 which is connected to the outputs of LEDs 74-78 and a display multiplex driver 96 which is connected to the outputs of LEDs 82-86.

Thus, in response to the digitized data signal input to the DATA input of microprocessor 50, microprocessor 50 controls drivers 92, 94 and 96 to control illumination of selective LEDs 74-78 and 82-86. In this regard, depending upon which LEDs are illuminated, an indication is provided as to the inhalation rapidity, that is, the rise time of inhalation, as displayed by display panel 70 and the inhalation flow rate peak as displayed by display panel 72. Numerical values can be provided adjacent transparent bars 80 and 88 of panels 70 and 72. Thus, measured values or quantities are compared to critical values required for good inhalation technique in microprocessor 50, and a score is computed and displayed by panels 70 and 72.

In use, after a mouthpiece 30 is installed on housing 14 of DPI simulator 12, START button 66 is depressed. ON/OFF timer circuit 62 thereafter maintains the power supply to microprocessor 50 for a predetermined amount of time, for example 30 seconds. To confirm the power on condition, displays 70 and 72 are turned on, indicating the lowest possible score, that is, one red LED 74 and one red LED 82 are illuminated. The patient then inhales rapidly and deeply through DPI simulator 12, in the same manner that a dry powder medicament inhaler is used.

Microprocessor 50 continually reads the digitized output of A/D converter 48, which represents the pressure drop due to inhalation flow. When the reading exceeds the digitized value corresponding to the start of inhalation, for example, 10 L/min, microprocessor 50 starts timing the rise time between two fixed values of an initial value and an ending value, for example, 10 L/min and 30 L/min. The rise time or inhalation rapidity is based on the difference between the ending value of 30 L/min and the initial value of 10 L/min, over time. Based on predetermined laboratory data of rise time versus respirable particle fraction, a table of scores was previously developed. Thereafter, LEDs 74-78 are selectively illuminated, based on these scores, with green bars 78 indicating shorter air flow rise times (higher inhalation rapidity).

An example of one possible scoring table and display scale for inhalation rapidity is as follows, starting measuring from the leftmost red LED 74:

| Display | Rise Time (sec) | Interpretation |
|---|---|---|
| 1st red | >0.765 | very poor |
| 2nd red | 0.681 to 0.765 | very poor |
| 1st yellow | 0.360 to 0.680 | poor |
| 2nd yellow | 0.201 to 0.359 | poor |
| 3rd yellow | 0.120 to 0.200 | marginal |
| 1st green | 0.081 to 0.119 | minimum acceptable |
| 2nd green | 0.060 to 0.080 | good |
| 3rd green | 0.045 to 0.059 | very good |
| 4th green | 0.033 to 0.044 | very, very good |
| 5th green | <0.033 | excellent |

In such case, indications of very poor, poor or marginal rise times mean that the patient must continue the training.

For every reading of the digitized output signal from A/D converter 48, microprocessor 50 also compares the most recent reading to the last highest reading. If the most recent reading is higher than the last highest reading, microprocessor substitutes the last highest reading as the reading for the flow rate peak. The last highest reading is displayed as a score on panel 72, with green bars 88 indicating higher flow rates. The following is an example of one possible scoring table and display scale for inhalation flow rate peak:

| Display | Peak Flow (L/min) | Interpretation |
|---|---|---|
| 1st red | <20 | very low peak flow |
| 2nd red | 21 to 25 | very low peak flow |
| 1st yellow | 26 to 30 | low peak flow |
| 2nd yellow | 31 to 35 | low peak flow |
| 3rd yellow | 36 to 40 | marginal peak flow |
| 1st green | 41 to 45 | acceptable peak flow |
| 2nd green | 46 to 50 | good peak flow |
| 3rd green | 51 to 55 | very good peak flow |
| 4th green | 56 to 60 | very, very good peak |
| | | flow |
| 5th green | >60 | excellent peak flow |

In such case, indications of very low, low or marginal peak flows mean that the patient must continue the training.

In the event that the patient inhales with ,a short breath, for example, less than 0.75 second, the scores are set to the lowest values on panels 70 and 72.

Thus, training device 10 fulfills the need to train dry powder inhaler users to achieve proper inhalation technique by measurement, comparison and display of a user's inhalation flow rate peak and inhalation rapidity. The measured quantities are compared to critical values required for good inhalation technique and a score is computed and displayed.

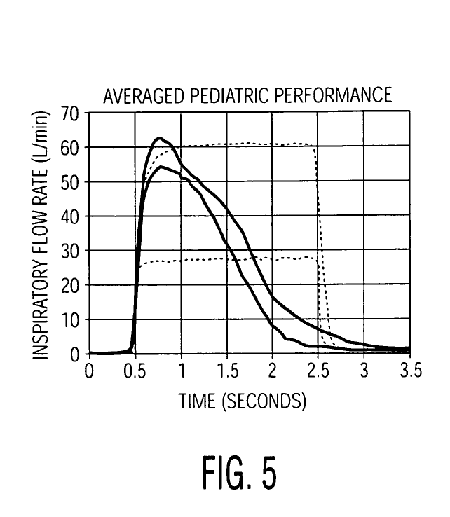

In clinical studies using training device 10, a total of 82 inhalation flow rate measurements were obtained from 54 adults with mild or severe asthma and from children between 5 and 12 years of age. Graphic plots of the data obtained from the clinical studies are shown in FIGS. 4 and 5 for the adults and children, respectively. All but one patient, whose rise time was 495 milliseconds (msec), demonstrated inhalation flow rate peaks greater than or equal to 53 L/min and rise times generally less than or equal to 70 msec. The generally square curves shown in broken lines in FIGS. 4 and 5 represent inhalation flow rates of 60 L/min and 28.3 L/min, which were generated by an in vitro testing device known as a “cascade impactor.” As shown, the inhalation flow rates and the rise times generated by the adults with mild and severe asthma and the children, were all within the target range.

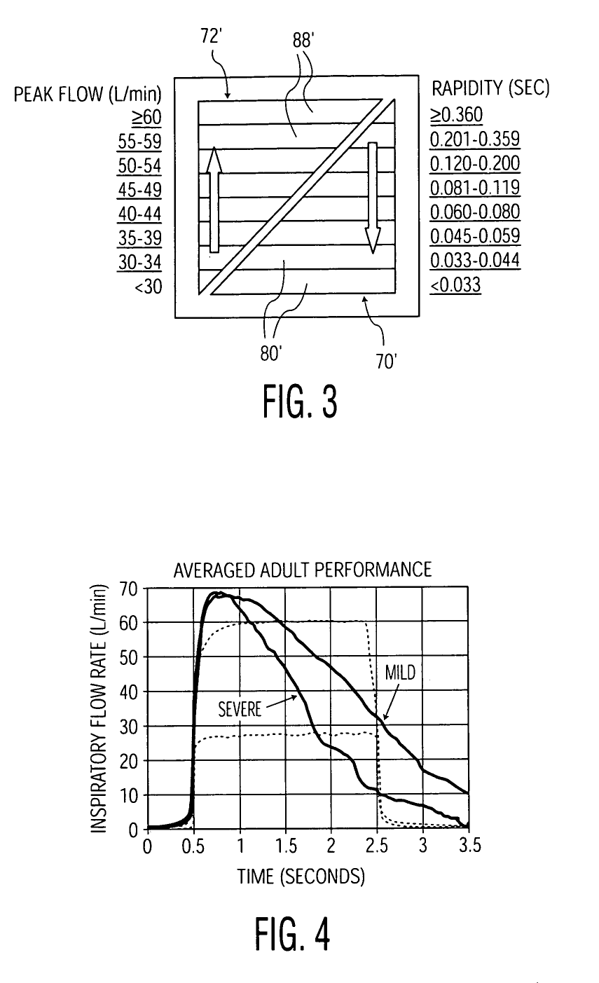

It will be appreciated that various modifications can easily be made to the present invention, within the scope of the claims herein. Thus, although inhalation rapidity display panel 70 and inhalation flow rate peak panel 72 are shown in FIGS. 1 and 2 as two linearly arranged panels, other arrangements can be provided. For example, each display panel 70′ and 72′ of FIG. 3 is represented by a triangular segment, with each triangular segment divided into eight transparent slices or bars 80′ and 88′, respectively, and with the scores printed adjacent each bar 80′ and 88′. In such case, the larger the triangular area that is illuminated on panels 70′ and 72′, the better the score. When a patient inhales rapidly and deeply on mouthpiece 30, for example, for at least 0.75 second, the LEDs on display panels 70′ and 72′ will move from a walking pattern to solid triangular patterns. Preferably, a patient should continue training until the triangular patterns on display panels 70′ and 72′ pass each other. The ideal score is shown by both triangular patterns being fully filled or lighted.

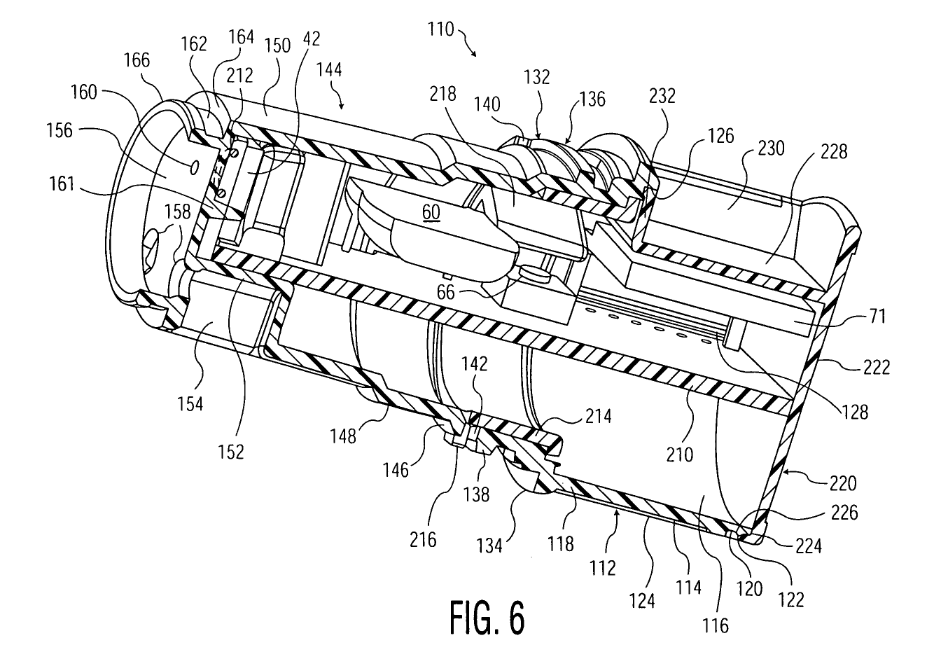

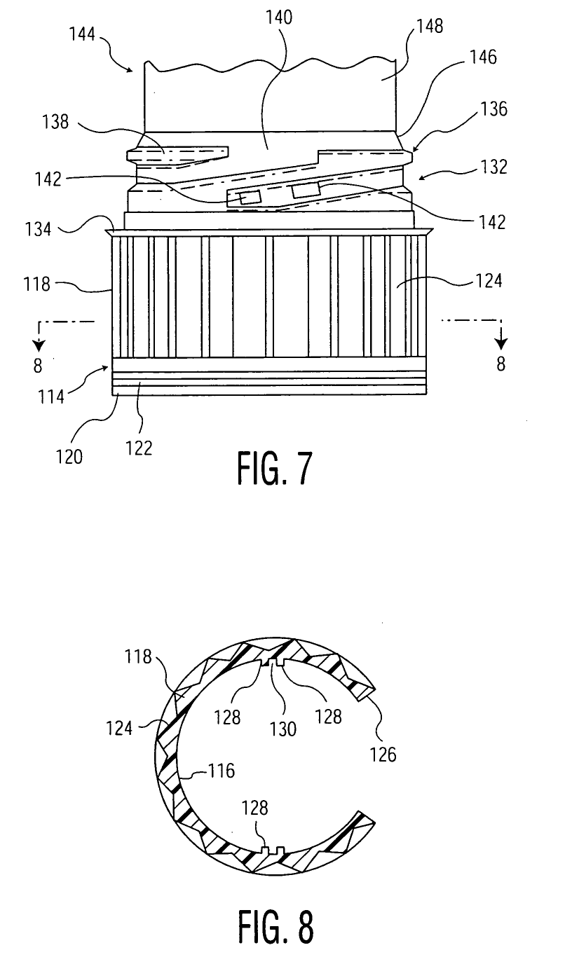

Referring now to FIGS. 6-8, a specific embodiment of a second training device 110 according to the present invention will now be described. Training device 110 differs from training device 10 in that all components are contained within the DPI simulator housing.

As shown therein, training device 110 includes a DPI simulator 112 having a single hollow housing 114 which is preferably of the same overall exterior dimensions and shape as those for simulator 12 of FIG. 1, and is preferably molded from a thermoplastic material. As with housing 14, housing 114 also includes a central axial bore 116 therein. Bore 116 is shown to increase in diameter toward the lower end, in accordance with the outer configuration of housing 114. This is provided so that the walls of housing 114 are of a uniform thickness. However, the present invention is not limited thereto, and central bore 116 can have a constant thickness throughout, with the walls of housing 114 varying in thickness, or any other suitable arrangement.

Housing 114 includes a lower annular wall 118 having an annular outer recessed area 120 formed at the lower end thereof, with an annular rib 122 formed on the outer surface of outer recessed area 120, slightly spaced above the lower edge of lower annular wall 118. The outer surface of lower annular wall 118 is preferably provided with a gripping surface 124 formed by undulations, knurling or the like, to enhance the gripping and rotation of housing 114.

A rectangular viewing opening 126 is formed in lower annular wall 118, and extends to the lower edge of lower annular wall 118 so as to be open thereat, the purpose for which will become readily apparent from the discussion hereinafter.

Further, two pairs of guide walls 128 are formed diametrically opposite to each other on the inner surface of lower annular wall 118, with each pair being offset from a center line of rectangular viewing opening 126 by about 90°. All of guide walls 128 are parallel to each other and extend in the same direction as the longitudinal axis of housing 114. Further, the guide walls 128 of each pair are positioned close to each other, with a small lengthwise gap 130 therebetween.

Housing 114 further includes an intermediate annular wall 132 of a lesser diameter than lower annular wall 118, and connected to the upper end of lower annular wall 118 by an outer annular shoulder 134. The outer surface of intermediate annular wall 132 is formed with a double helical cam track 136. As is apparent, the walls 138 that form double helical cam track 136 have a substantially square cross-section. Further, the entry 140 to each cam track 136 is formed as a vertical drop zone before rotation can begin, thus ensuring accurate registry of the closure cap and thereby, accurate operation of training device 110, in the same manner as corresponding structure of the inhaler of Published International Patent Application WO 97/30743.

Two slightly spaced apart, small rectangular openings 142 are formed in one cam track 136, diametrically opposite to the position of viewing opening 126, the purpose for which will be apparent from the description which follows.

Housing 114 further includes an upper annular wall 144 of a lesser diameter than intermediate annular wall 132, and connected to the upper end of intermediate annular wall 132 by an outer annular shoulder 146. Upper annular wall 144 includes a lower annular section 148 connected to intermediate annular wall 132 through annular shoulder 146 and an upper annular section 150 of a lesser diameter than lower annular section 148 and connected to an upper end of lower annular section 148. The upper end of upper annular section 150 is formed with two recessed wall sections 152 extending radially inward therefrom and in general diametric relation to the position of viewing opening 126, thereby forming outer air inlet channels 154 to provide secondary air flow, as will be apparent from the description which follows.

The upper end of upper annular section 150 is closed off by a circular top wall 156 having two outer openings 158 therein which are in alignment with outer air inlet channels 154 to receive outside air from outer air inlet channels 154 during inhalation. In addition, circular wall 156 includes a pressure sensing opening 160 positioned in a plane that generally bisects openings 158. Pressure sensing opening 160 is the opening through which air pressure is detected when a patient inhales. Specifically, pressure sensing opening 160 communicates with a pressure sensor within housing 114, as will be described hereinafter. In addition, two pins 161 also extend down from the inner surface of top wall 156 in spaced relation from recessed wall sections 152.

An annular mouthpiece securing wall 162 is formed on the upper surface of circular top wall 156, spaced slightly inwardly from the peripheral edge thereof. As a result, an annular ledge 164 is formed on the upper surface of circular top wall 156, outwardly of annular mouthpiece securing wall 162. Further, an annular lip 166 extends outwardly in the radial direction from the upper end of annular mouthpiece securing wall 162.

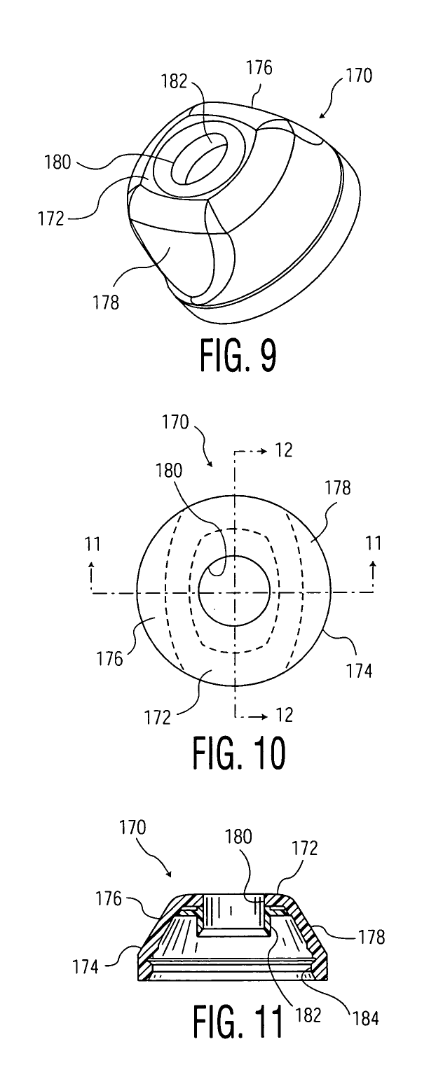

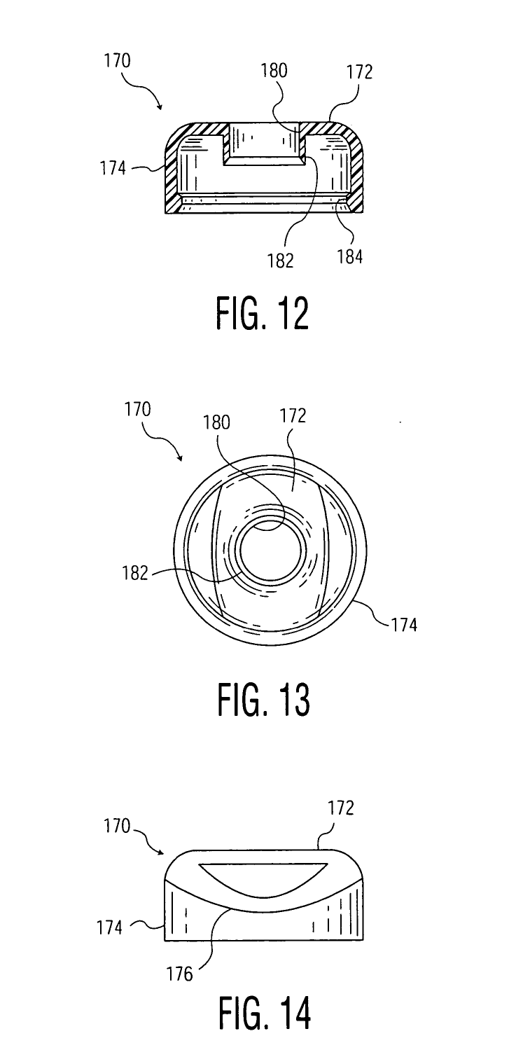

A mouthpiece 170, as shown in FIGS. 9-14, is secured to the upper end of housing 114. Mouthpiece 170 includes a generally rectangular top wall 172 with an annular side wall 174 extending downwardly from the periphery of top wall 172. Because top wall 172 has a generally rectangular configuration and because of the annular configuration of side wall 174, upper portions at opposite sides 176 and 178 of side wall 174 corresponding to the lengthwise sides of top wall 172 slope upwardly in a diverging manner toward each other. The lips of a user of the device are placed on sides 176 and 178 during inhalation. Of course, since the user's mouth is placed over mouthpiece, the various edges thereof are rounded. A central opening 180 is centrally formed in top wall 172, and an annular stub tube 182 is formed at the lower surface of top wall 172 in surrounding relation to opening 180.

In order to secure mouthpiece 170 to annular mouthpiece securing wall 162, the lower end of side wall 174 has a circular or annular shape. At the inner surface of this lower end of side wall 174, there is formed an annular V-shaped projection or rib 184 which extends inwardly in the radial direction. When mouthpiece 170 is positioned on annular mouthpiece securing wall 162 and pressed down thereon, annular lip 166, due to resilience of the plastic pieces, rides over V-shaped projection 184, so that V-shaped projection 184 retains mouthpiece 170 on annular mouthpiece securing wall 162. In such position, the lower edge of side wall 174 sits on annular ledge 164.

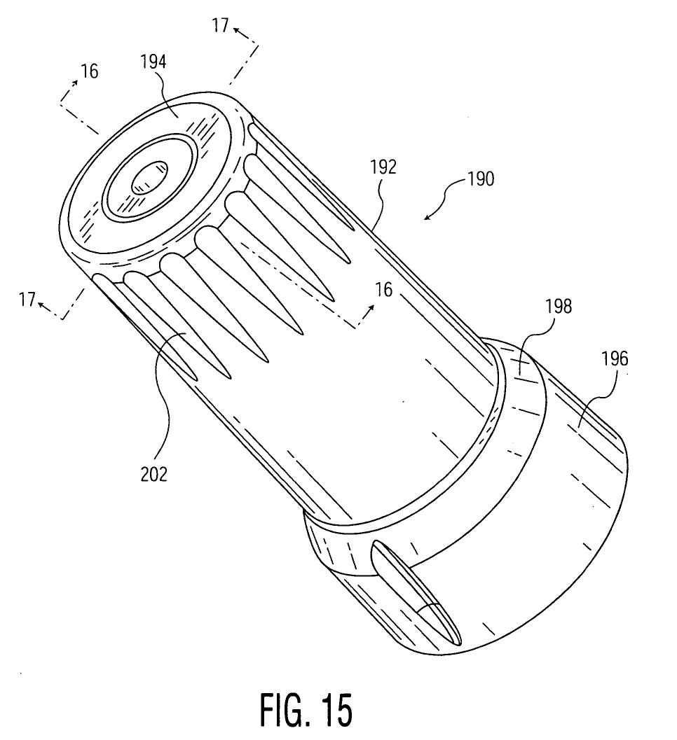

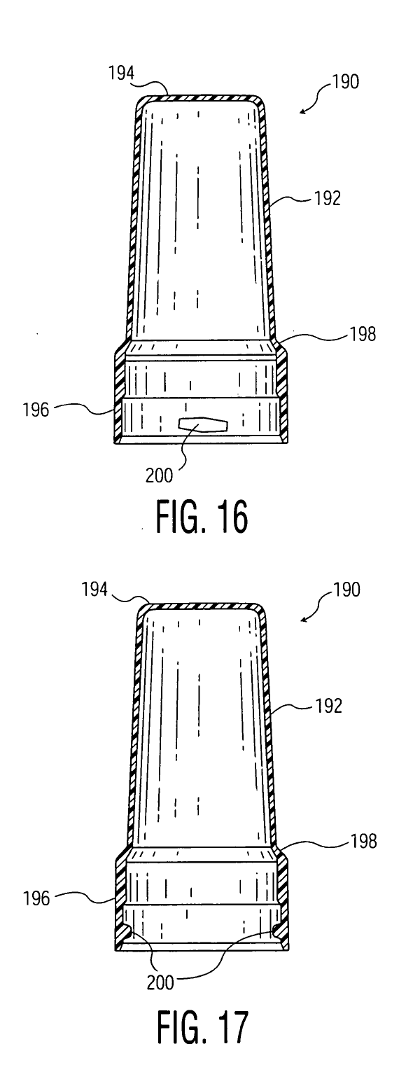

Referring now to FIGS. 15-17, a closure cap 190 of training device 110 is provided as a closure for DPI simulator 112, and at the same time, functions to actuate the start button or switch for the circuitry. Specifically, closure cap 190 includes an upper elongated annular covering wall 192 which is closed at its upper end by a generally circular top wall 194. A lower annular securing skirt 196 of a larger diameter than annular covering wall 192, is secured to the lower end of annular covering wall 192 through an annular frusto-conical connector 198. The lower end of annular securing skirt 196 is open. Further, the inner diameter of lower annular securing skirt 196 is slightly larger than the outer diameter of intermediate annular wall 132 of housing 114 so as to fit thereover.

In order to secure closure cap 190 onto housing 114, and particularly, in covering relation to mouthpiece 170, two helix cams 200 are formed in diametrically opposite relation on the inner surface of lower annular securing skirt 196. Thus, when closure cap 190 is inserted over housing 114 and mouthpiece 170, cams 200 of closure cap 190 initially vertically drop in entry 140 and then threadedly engage with double helical cam track 136 on intermediate annular wall 132, until the lower edge of lower annular securing skirt 196 seats on outer annular shoulder 134.

It is noted that cams 200 and cam track 136 are provided in place of conventional screw threads. This is because, with conventional screw threads, cap 190 may be prematurely pulled off due to the tolerance of the threads. As a result, training device 110 may not be operated correctly. However, with cams 200 and cam track 136 having walls 138 of a square cross-section, numerous advantages are achieved, including preventing premature opening of cap 190, ease of use, and ensuring proper location at all times of the rotational positions of the parts of training device 110. Thus, cap 190 cannot engage with housing 114 until cams 200 are fully engaged in cam track 136.

It will be appreciated that the outer diameter of lower annular securing skirt 196 is substantially identical with the outer diameter of lower annular wall 118 of housing 114 to provide a relative smooth, continuous appearance. In order to aid in the removal and closing of closure cap 190, the outer surface of annular covering wall 192 is formed with a gripping surface 202 formed by undulations, knurling or the like, to enhance the gripping and rotating of closure cap 190.

Closure cap 190 also serves to actuate the start button for the circuitry. Specifically, cam 200 engages projection 216 in order to move internal ring 214 relative to housing 314 when the closure cap 190 is fully installed. Although two cams 200 are shown, only one needs be provided if the closure cap can be installed from only one position.

Referring back to FIG. 6, a planar circuit board 210 is mounted in housing 114. Specifically, opposite edges of circuit board 210 fit within and are restrained by guide walls 128. The upper end of circuit board 210 is also restrained by recessed wall sections 152 and pins 161.

All electronic components are mounted on circuit board 210. Specifically, pressure sensor or pressure transducer 42 is mounted on circuit board 210 immediately below pressure sensing opening 160, and a sealing gasket 212 made from a low durometer polymer, is provided therearound, so that the pressure drop through pressure sensing opening 160 is accurately detected by pressure sensor 42. The output from pressure sensor 42 is supplied through microprocessor 50 which includes an integral analog-to-digital converter. The crystal 54, voltage detector 56, test/calibrate switch 58, on/off timer 62 and voltage regulator 64 are not shown for the sake of simplicity of the drawing.

In addition, battery 60, such as a single low cost 3 volt lithium “coin” cell, momentary start button 66 and a liquid crystal display (LCD) panel 71 which is a combination of both display panels 70 and 72 of the first embodiment, are also mounted on circuit board 210. Momentary start button 66 applies power to the electronic circuitry from battery 60. In this regard, an internal ring 214 is rotatably mounted along the inner surface of intermediate annular wall 132 and includes two projections 216 which extend out from rectangular openings 142 of intermediate annular wall 132. Rotatable displacement of internal ring 214 is caused by sliding movement of projections 216 within rectangular openings 142, the limits of such movement being restricted by the ends of rectangular openings 142. Displacement of projections 216 within rectangular openings 142 is caused by a cam 200 on closure cap 190 during removal of and securement of closure cap 190 on housing 114.

Internal ring 214 includes an actuation projection 218 extending substantially radially inward from the inner wall thereof, in opposing relation to momentary start button 66. When closure cap 190 is removed, a cam 200 engages one projection 216 in order to rotatably slide internal ring 214 relative to housing 114, and thereby cause actuation projection 218 to move into engagement with momentary start button 66 in order to supply power to the circuitry. This is similar to the normal dispensing operation of the dry powder medicament inhaler of the aforementioned patents. As with the previously described embodiment of the training device, power will normally be discontinued electronically after a preselected time, for example 30 seconds. During this time, a patient who inhales through mouthpiece 170 will cause a pressure differential during inhalation to be sensed by pressure sensor 42, and the results will be displayed on display panel 71, as discussed above.

When the patient has completed the test inhalation or when the predetermined activation time limit has expired, it will be necessary for closure cap 190 to be reinstalled. At this time, a cam 200 engages the other projection 216 to cause reverse rotation of internal ring 214, thereby moving actuation projection 218 away from momentary start button 66.

Further, a lower end cap 220 is mounted to the lower end of housing 114 for retaining circuit board 210 in position, for protectively closing the open end of housing 114 and for protecting display panel 71. End cap 220 includes a circular bottom wall 222 and a short annular upstanding wall 224 which fits in annular recessed area 120, and which has an annular recess 226 at the inner surface thereof for receiving annular rib 122 in order to hold end cap 220 on housing 114. In addition, annular upstanding wall 224 is cut-away over an arc of about 60°. An upstanding view window 228 extends upwardly from bottom wall 222 over an arc of about 50°, with angled side walls 230 extending from opposite ends of view window 228 to opposite ends of upstanding wall 224. A top wall 232 also extends radially outwardly from the upper edge of view window 228, and is connected at opposite sides thereof with side walls 230. When end cap 220 is positioned on housing 114, view window 228 is positioned directly in front of display panel 71. View window 228 is formed from a transparent or translucent material, so that display panel 71 can be viewed therethrough.

Thus, with mouthpiece 170 in place, a plenum is formed by the interior volume of mouthpiece 170, and during inhalation through mouthpiece 170, an area of reduced air pressure is generated within the plenum because of restricted airflow through openings 158 and 160. Pressure sensor 42 responds to this decreased pressure with a voltage signal which is applied to the analog-to-digital converter integrated with microprocessor 50. Peak inspiratory flow rates and rise times are derived in the previously described manner, and are displayed, such as in the above-noted triangular segment format, by liquid crystal display panel 71.

This embodiment does not provide a mechanically adjustable flow calibration feature, as with set screw 26 of the first embodiment. However, such calibration can be performed electronically. For example, following assembly of the device, a vacuum source can be connected to mouthpiece 170, to generate a constant airflow through air inlet channels 154 and fixed openings 158 and 160. A 60 L/min flow rate has been found satisfactory for this purpose. The program in microprocessor 50 is such that the first pressure drop experienced by pressure sensor 42, for a predetermined time such as 3 seconds, causes the electronics to enter a calibration mode wherein the known constant airflow rate is used to generate a factor which is stored into a memory and used to compensate for small variations in fixed orifice dimensions of openings 158 and 160, resulting from normal tolerances of the manufacturing procedure. As long as the pressure drops for the known flow are within an allowable range, for example, ±1 kPa, the trainer can be adjusted electronically.

Training device 110 of FIGS. 6-17 has the advantage of having a lower manufacturing cost than training device 10 of FIGS. 1-5. Specifically, with circuit board 210 being within training device 110, no conduit is required. In such case, the power switch or momentary start button 66 is actuated by the opening of closure cap 190, mimicking the action of the actual inhaler. The present embodiment also provides for a low power device, with the use of a single low cost 3 volt lithium coin cell. Also, rather than using an LED bar graph, a liquid crystal display (LCD) 71 is used.

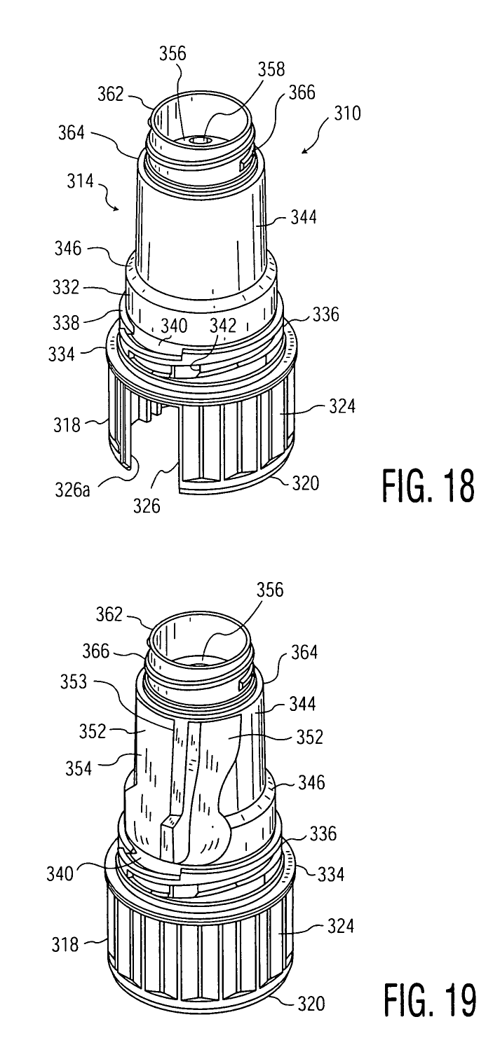

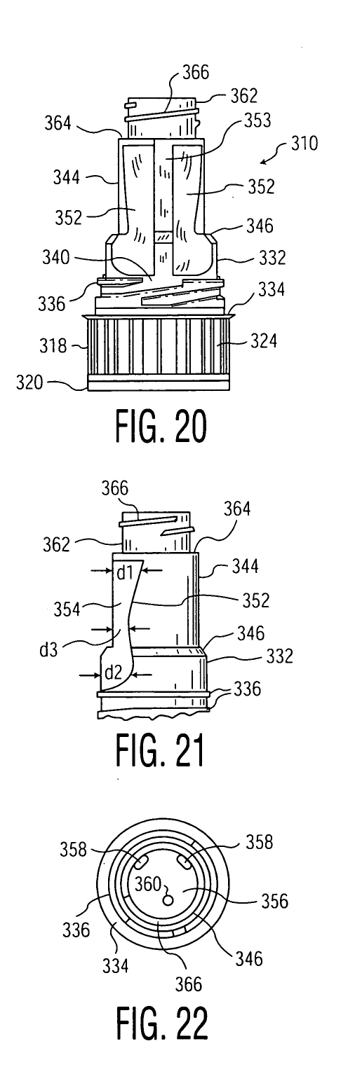

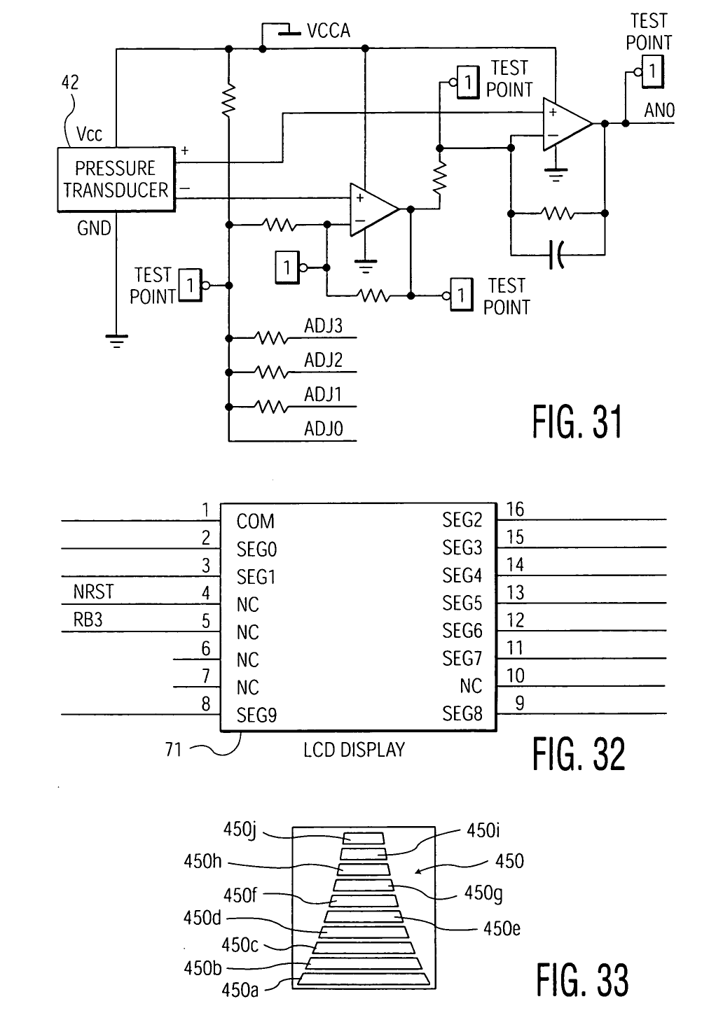

Referring now to FIGS. 18-33, a preferred embodiment of another training device 310 according to the present invention will be described. Training device 310 is similar in many respects to training device 110 and therefore, certain common components will be referenced by the same numerals, but incremented by 200. As with training device 110, all components of training device 310 are contained within the DPI simulator housing.

As shown in FIGS. 18-22, single hollow housing 314 is preferably of the same overall exterior dimensions and shape as those for simulator 12 of FIG. 1 and single hollow housing 114 of FIG. 6, and is conveniently molded from a thermoplastic material. As with housing 114, housing 314 also includes a central axial bore therein which increases in diameter toward the lower end, in accordance with the outer configuration of housing 314.

Housing 314 includes a lower annular wall 318 having an annular outer recessed area 320 formed at the lower end thereof. As with housing 114, an annular rib (not shown) is preferably formed on the outer surface of outer recessed area 320, slightly spaced above the lower edge of lower annular wall 318. The outer surface of lower annular wall 118 is preferably provided with a gripping surface 324 formed by undulations, knurling or the like, to enhance the gripping and rotation of housing 314.

A rectangular viewing opening 326 is formed in lower annular wall 318, and extends to the lower edge of lower annular wall 318 so as to be open thereat, the purpose for which will become readily evident from the discussion hereinafter. Rectangular viewing opening 326 includes slotted side edges 326a.

Housing 314 further includes an intermediate annular wall 332 of a lesser diameter than lower annular wall 318, and connected to the upper end of lower annular wall 318 by an outer annular shoulder 334. The outer surface of intermediate annular wall 332 is formed with a double helical cam track 336. As is apparent, the walls 338 that form double helical cam track 336 have a substantially square cross-section. Further, the entry 340 to each cam track 336 is formed as a vertical drop zone before rotation can begin, thus ensuring accurate registry of the closure cap and, thereby, accurate operation of training device 310, in the same manner as corresponding structure of the inhaler of Published International Patent Application WO 97/30743.

A single small rectangular opening 342 is formed in one cam track 336, directly above one side edge of viewing opening 326, the purpose for which will be apparent from the description which follows. Alternatively, rectangular opening 342 can extend in the axial direction along both cam tracks 336.

Housing 314 further includes an upper annular wall 344 of a lesser diameter than intermediate annular wall 332, and connected to the upper end of intermediate annular wall 332 by an outer annular shoulder 346. Upper annular wall 344 is connected to intermediate annular wall 332 through annular shoulder 346. The entire axial length of upper annular wall 344 is formed with two recessed wall sections 352 extending radially inward therefrom and in general diametric relation to the position of viewing opening 326, thereby forming outer air inlet channels 354 to provide secondary air flow, as will be apparent from the description which follows. Recessed wall sections 352 also extend into intermediate annular wall 332 to a position just above cam tracks 336. Recessed wall sections 352 are separated by an axially extending separating wall 353 that extends the entire axial length of upper annular wall 344 and extends into intermediate annular wall 332 to a position just above cam tracks 336. As shown best in FIG. 21, the radial depth “d1” of recessed wall sections 352 at the upper end and the radial depth “d2” at the lower end are greater than the radial depth “d3” at an intermediate portion which, as will be understood from the discussion hereafter, which results in a venturi effect.

The upper end of upper annular wall 344 is closed off by a circular top wall 356 having two outer openings 358 therein which are in alignment with outer air inlet channels 354 to receive outside air from outer air inlet channels 354 during inhalation. In addition, circular wall 356 includes a pressure sensing opening 360 positioned in a plane that generally bisects openings 358. Pressure sensing opening 360 is the opening through which air pressure is detected when a patient inhales. Specifically, pressure sensing opening 360 communicates with a pressure sensor within housing 314, as will be described hereinafter.

An annular mouthpiece securing wall 362 is formed on the upper surface of circular top wall 356, spaced slightly inwardly from the peripheral edge thereof. As a result, an annular ledge 364 is formed on the upper surface of circular top wall 356, outwardly of annular mouthpiece securing wall 362. Further, a helical thread 366 extends outwardly in the radial direction from the upper end of annular mouthpiece securing wall 362.

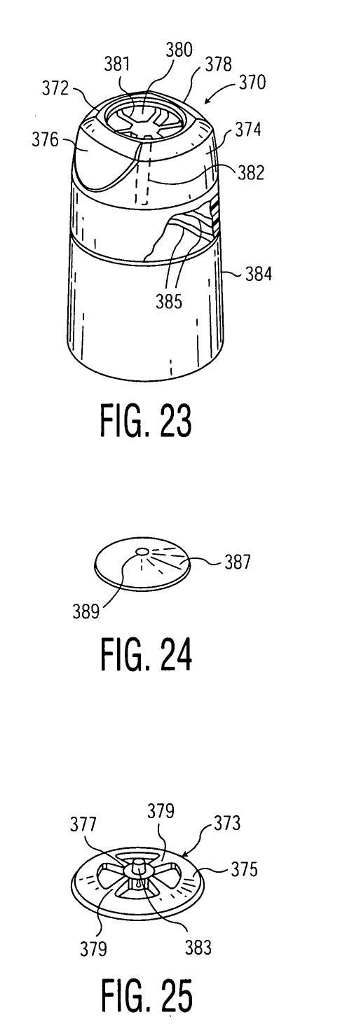

A mouthpiece 370, as shown in FIG. 23, is secured to the upper end of housing 314. Mouthpiece 370 includes a generally rectangular top wall 372 with an annular side wall 374 extending downwardly from the periphery of top wall 372. Because top wall 372 has a generally rectangular configuration and because of the annular configuration of side wall 374, upper portions at opposite sides 376 and 378 of side wall 374 corresponding to the lengthwise sides of top wall 372 slope upwardly in a diverging manner toward each other. The lips of a user of the device are placed on sides 376 and 378 during inhalation. Of course, since the user's mouth is placed over mouthpiece, the various edges thereof are preferably rounded. A central opening 380 is centrally formed in top wall 372. Cruciform walls 381 are formed at opening 380 and extend down therefrom, with a hollow stub post 382 formed at the center of cruciform walls 381 and extending down therefrom in the axial direction of mouthpiece 370, substantially to the lower end thereof.

Mouthpiece 370 is fixed to a hollow tube 384 having a length such that when mouthpiece 370 and hollow tube 384 are fit over housing 314, the lower edge of hollow tube 384 rests on outer annular shoulder 346. Specifically, in order to secure mouthpiece 370 to annular mouthpiece securing wall 362, the inner surface of hollow tube 384, at the upper end thereof, includes a helical thread 385 which extends radially inwardly from the inner surface of hollow tube 384, for mating with helical thread 366. When threads 366 and 385 are fully mated with each other, the lower edge of hollow tube 384 sits on annular shoulder 346. It will therefore be appreciated that mouthpiece 370 should be a single patient-use component to prevent cross transmission of disease and contamination of training device 310. Thus, a new mouthpiece 370 will be used for each patient, merely by unscrewing the old mouthpiece and disposing of the same, and threading a new mouthpiece 370 in place thereof.

In order to aid in preventing contamination, a one-way check valve arrangement is provided in hollow tube 384 at the upper end thereof immediately above helical thread 385. Specifically, the one-way valve arrangement includes a circular retainer 373 (FIG. 25) secured in sealing relation, for example, by welding, to the inner surface of hollow tube 384 at the upper end thereof immediately above helical thread 385. Circular retainer 373 includes an annular wall 375, the outer periphery of which is secured to hollow tube 384, a center hub 377 within annular wall 375 and a plurality of radially directed ribs 379 equiangularly spaced apart and which interconnect center hub 377 with annular wall 375. Although four ribs 379 are shown, the present invention is not limited thereby. Preferably, the height of ribs 379 is greatest at center hub 377 so that the upper surfaces thereof slope down toward annular wall 375. A stub post 383 extends upwardly from the center of the upper surface of center hub 377.

The one-way valve arrangement further includes a circular valve flap 387 (FIG. 24) having a center opening 389. Valve flap 387 is fit on the upper surface of circular retainer 373 so as to cover center hub 377, ribs 379 and the radially inner portion of annular wall 375. Specifically, stub post 383 extends through center opening 389 and is received in the lower end of hollow stub post 382 to lock valve flap 387 in position.

Thus, when a patient inhales through mouthpiece 370, the suction causes valve flap 387 to raise up, thereby permitting air to pass upwardly through the gaps between ribs 379. However, if the patient exhales, the force of the exhalation forces valve flap 387 into sealing relation with circular retainer 373 in order to prevent contamination of training device 310.

A closure cap which is similar to closure cap 190 of training device 110, shown in FIGS. 15-17, is provided as a closure for mouthpiece 370, and at the same time, functions to actuate the start button or switch for the circuitry. As will be understood from the discussion hereafter, actuation occurs by helix cams 200 which also serve to secure the closure cap onto housing 314, by initially vertically dropping in entry 340 and then threadedly engaging with double helical cam track 336 on intermediate annular wall 332, until the lower edge of the lower annular securing skirt of the closure cap seats on outer annular shoulder 334.

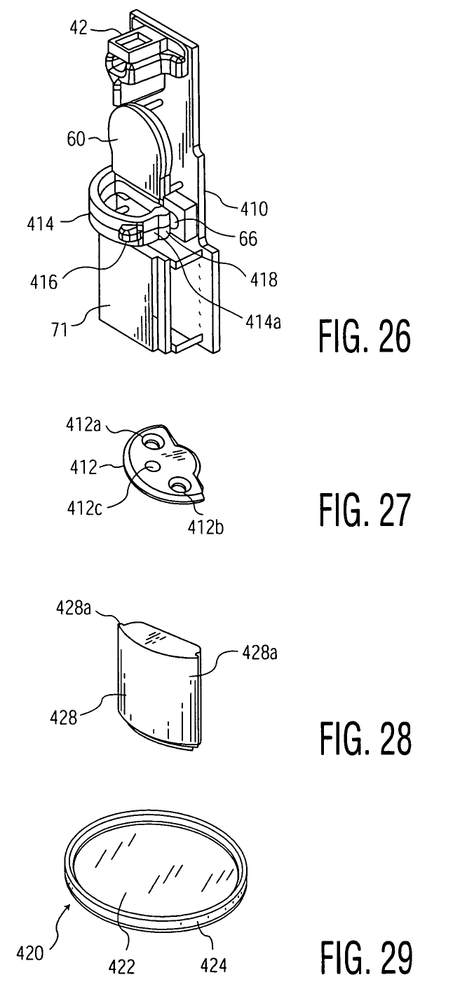

Referring now to FIG. 26, a planar circuit board 410 is mounted in housing 314. Specifically, opposite edges of circuit board 410 fit within and are restrained by guide walls (not shown) in housing 314.

All electronic components are mounted on circuit board 410. Specifically, pressure sensor or pressure transducer 42 is mounted on circuit board 410 immediately below pressure sensing opening 360, and a sealing gasket 412 (FIG. 27) made from a low durometer polymer, is provided therearound, so that the pressure drop through pressure sensing opening 360 is accurately detected by pressure sensor 42. Sealing gasket 412 includes openings 412a, 412b and 412c corresponding in alignment with openings 358 and 360. The output from pressure sensor 42 is supplied through microprocessor 50 which includes an integral analog-to-digital converter. Some components are not shown for the sake of simplicity of the drawing.

In addition, battery 60, such as a single low cost 3 volt lithium coin cell, momentary start button 66 and a liquid crystal display panel 71, which is a combination of both display panels 70 and 72 of the first embodiment, are also mounted on circuit board 410. Momentary start button 66 applies power to the electronic circuitry from battery 60.

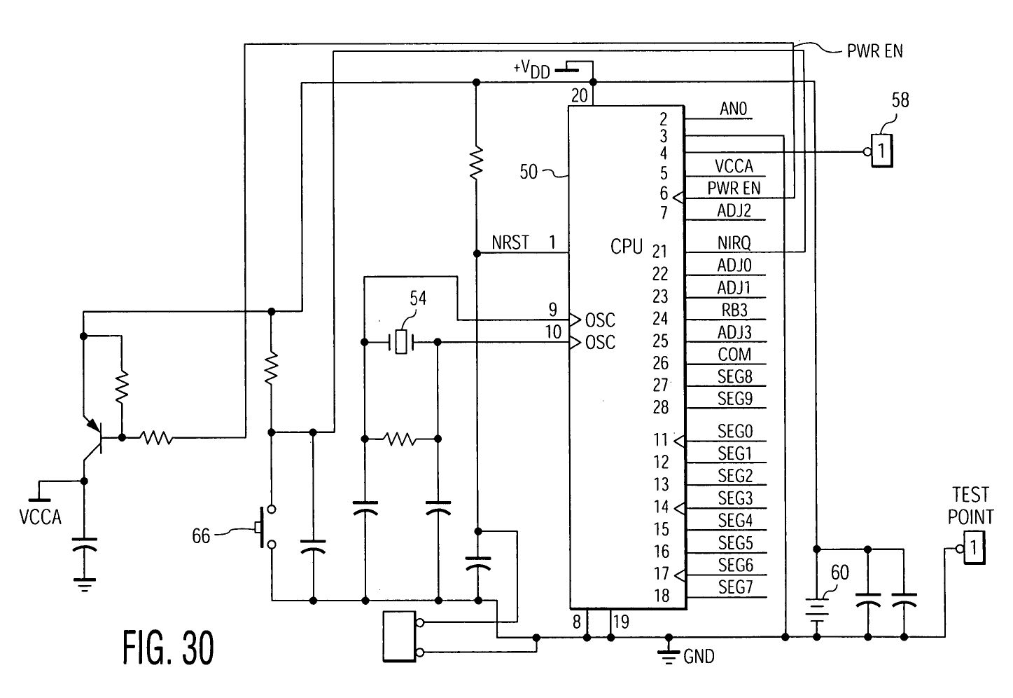

The electronic circuitry of circuit board 410 is shown in more detail in FIGS. 30-32.

An internal ring 414 is pivotally mounted in a cantilever manner on circuit board 410. A projection 416 is mounted on the outer surface of internal ring 414 near the free end 414a thereof and extends out from rectangular opening 342 of intermediate annular wall 332. Rotatable displacement of internal ring 414 is caused by sliding movement of projection 416 within rectangular opening 342. Displacement of projection 416 within rectangular opening 342 is caused by either helix cam 200 on closure cap 190 during removal of and securement of closure cap 190 on housing 314.

Internal ring 414 includes an actuation projection 418 extending substantially radially inward from the inner wall thereof, in opposing relation to momentary start button 66. When closure cap 190 is removed, either helix cam 200 engages projection 416 in order to move internal ring 414 relative to housing 314, and thereby cause actuation projection 418 to move into engagement with momentary start button 66 in order to supply power to the circuitry. This is similar to the normal dispensing operation of the TWISTHALER dry powder medicament inhaler. As with the previously described embodiment of the training device, power will normally be discontinued electronically after a preselected time, for example, two minutes. During this time, a patient who inhales through mouthpiece 370 will cause a pressure differential to be sensed by pressure sensor 42, and the results will be displayed on display panel 71, as discussed above.

When the patient has completed the test inhalation or when the predetermined activation time limit has expired, it will be necessary for closure cap 190 to be reinstalled. At this time, the same helix cam 200 engages projection 416 again in order to move internal ring 414 relative to housing 314, and thereby cause actuation projection 418 to move into engagement once again with momentary start button 66 in order to shut off power to the circuitry.

A transparent curved lens 428 (FIG. 28) having the same dimensions as rectangular viewing opening 326, is mounted within rectangular viewing opening 326. Specifically, lens 428 includes side ribs 428a at the side edges thereof which slide within slotted side edges 326a so as to position lens 428 in covering relation to rectangular viewing opening 326. In this position, the displayed results from display panel 71 can be viewed therethrough. A lower end cap 420 (FIG. 29) is mounted to the lower end of housing 314 for retaining lens 428 and circuit board 410 in position, for protectively closing the open end of housing 314 and for protecting display panel 71. End cap 420 includes a circular bottom wall 422 and a short annular upstanding wall 424 which is secured to the lower end of housing 314.

Thus, with mouthpiece 370 in place, a plenum is formed by the interior volume of mouthpiece 370, and during inhalation through mouthpiece 370, a reduced air pressure is generated within the plenum because of restricted airflow through openings 358 and 360. Because of the reduced radial depth d2 of recessed wall sections 352 at the intermediate portion, a venturi effect results when the air is sucked up through openings 358. Pressure sensor 42 responds to this decreased pressure with a voltage signal which is applied to the analog-to-digital converter integrated with microprocessor 50. Peak inspiratory flow rates and rise times are derived in the previously described manner, and are displayed, such as in the above-noted triangular segment format, by liquid crystal display panel 71.

As with training device 110, training device 310 does not provide a mechanically adjustable flow calibration feature, as with set screw 26 of the first embodiment training device 10. However, such calibration can be performed electronically in the same manner as described above in regard to training device 110.

For an example of a useful training device 310, the range of measurement of the inspiratory air flow rate can be between 0 and 75 liters per minute (L/min) with a range of 0 to 40 inches of water (0 to 10 Kpa) internal pressure differential (or pressure drop), and the specifications can include a ±5% variation of temperature, humidity and battery life time, and a resolution of 1 L/min. Preferably, the response time will be less than 4 msec and the sample rate is less than 4 msec, with a warm up time on powering up being less than 100 msec. In this case, the resistance to air flow is desirably the same as that for a medicament inhaler, for example 24 inches of water (6 Kpa) pressure differential at 60 to 70 L/min air flow and 6 inches of water (1.5 Kpa) internal pressure differential at 30 L/min air flow. Housing 314 should contribute a pressure differential of 5.5 to 5.9 Kpa and mouthpiece 370 should contribute a pressure differential less than 0.6 Kpa. Preferably, the training device 310 will provide a minimum of 2,000 measurement and display cycles or have at least a two year battery life with 1,000 measurement and display cycles.

As shown in FIG. 33, training device 310 provides a single bar graph display 450 on display panel 71 as to whether the inhalation is acceptable. The display 450 is a function of both inhalation rapidity and inhalation flow rate peak. Specifically, the display 450 includes ten color bars 450a-450j, the lowest bar 450a being red, the next three bars 450b-450d being yellow and the top six bars 450e-450j being green, although the present invention is not limited thereto. Acceptable scores occur when the red bar 450a, the three yellow bars 450b-450d and any one or more of green bars 450e-450j are illuminated.

In one embodiment, when training device 310 is off, room lighting will cause all of the bars to appear to be illuminated. Then when power is initially applied to training device 310, bars 450b-450j momentarily will appear black, then begin to blink. Alternatively, if the inhalation display is shown by blinking bars, all of the bars can be initially displayed in a non-blinking state.

In operation, closure cap 190 is removed from housing 314. When closure cap 190 is removed, one helix cam 200 engages projection 416 in order to move internal ring 414 relative to housing 314, and thereby cause actuation projection 418 to move into engagement with momentary start button 66 in order to supply power to the circuitry. At this time, display 450 will be black except for the flashing of red bar 450a. However, if there is no activity for a period of two minutes or more, or after a predetermined number of uses, training device 310 turns off by reason of an ON/OFF timer function which is programmed into microprocessor 50.

Then, a new mouthpiece 370 is affixed onto housing 314, and the patient inhales through mouthpiece 370 in the same manner as when using the medicament inhaler for which he or she is being trained. Microprocessor 50 continually reads the digitized output of its internal A/D converter, which represents the pressure drop due to inhalation flow. Based on these readings, an individual score of one to ten is determined for inhalation rapidity (rise time) and a separate individual score of one to ten is determined for inhalation peak flow rate. The scores are then combined to determine which of bars 450a-450j to illuminate.

Specifically, if either of the scores is four or less, the lower score is displayed, provided that the flow rate at 0.5 second from the start of inhalation is greater than 20 L/min. In such case, the display indicates that the patient has unsuccessfully performed the inhalation procedure.

If both of the two scores are five or higher, the two scores are averaged and truncated, that is, rounded down to the lowest whole number, to form a combined score. The combined score is displayed, provided that the flow rate at 0.5 second from the start of inhalation is greater than 20 L/min. In such case, the display indicates that the patient has successfully performed the inhalation procedure.

If the flow rate is equal to or less than 20 L/min after 0.5 second, the combined score is set to one, so that only the lowest red bar 450a is displayed. In such case, the display indicates that the patient has unsuccessfully performed the inhalation procedure.

As an alternative, in addition to the appropriate bars being displayed, all of the displayed bars can blink on and off, or only the uppermost bar can blink on and of.

A chart showing a useful rating arrangement is as follows:

| | Peak Flow | Rise Time |
|---|---|---|
| Score | (Liter/Minute) | (Second) |
| 10 | >60 | <0.050 |
| 9 | 47-60 | 0.050-0.080 |
| 8 | 44-47 | 0.080-0.090 |
| 7 | 41-44 | 0.090-0.100 |
| 6 | 38-41 | 0.100-0.110 |
| 5 | 35-38 | 0.110-0.120 |
| 4 | 30-35 | 0.120-0.300 |
| 3 | 25-30 | 0.300-0.500 |
| 2 | 20-25 | 0.500-0.765 |
| 1 | <20 | >0.765 |

As an example, if the rise time equals 0.150 second and the peak equals 62 L/min, with a flow reate at 0.5 second from the start of inhalation greater than 20 L/min the individual scores would be 4 and 10. Since one of the scores is 4 or less, only the bars 450a-450d would be illuminated, that is, one red bar 450a and three yellow bars 450b-450d, showing an unacceptable inhalation.

As another example, if the rise time equals 0.085 second and the peak equals 37 L/min, with a flow rate at 0.5 second from the start of inhalation above 20 L/min, the individual scores would be 8 and 5. The combined score would be a truncation of the average. Since the average of the scores is 6.5, this would be truncated to a score of 6, so that the lowest six bars 450a-450f would be illuminated, that is, one red bar 450a, three yellow bars 450b-450d and two green bars 450e and 450f, indicating an acceptable inhalation.

As still another example, if the rise time equals 0.030 second and the peak equals 62 L/min with a flow rate at 0.5 second from the start of inhalation less than 20 L/min, the individual scores would be 10 and 10. However, since the flow rate at 0.5 second was below the minimum requirement of 20 L/min, only the bottom red bar 450a would be illuminated.

According to clinical studies, 97% of adults and 98% of children over five years of age can achieve a 0.075 second rise time (score of 9) after training. Further, 98% of adults and 100% of children over five years of age can exceed a 46 L/min peak flow rate (score of 8) after training. If the flow rate is 20 L/min or above at 0.5 second from start of inhalation, these scores would result in a combined score or 8 which would result in the illumination of the lower eight bars 450a-450h, that is, red bar 450a, the three yellow bars 450b-450d and the four green bars 450e-450h. Therefore, it is not difficult to achieve or exceed the minimum requirements which give individual scores of at least 5, after training.

After the testing is completed, mouthpiece 370 is removed and discarded in order to prevent contamination and transmission of disease, and closure cap 190 is reinstalled over housing 314. Preferably, the dimensions of closure cap 190 and the mouthpiece 370 can be made such that the cap cannot be reinstalled if a mouthpiece 370 is present on the housing. Upon reinstallation of the cover cap, a helix cam 200 engages projection 416 in order to move internal ring 414 relative to housing 314, and thereby cause actuation projection 418 to move into engagement with momentary start button 66 to provide a signal to stop power to the circuitry.

Once an initial inhalation test is performed and the score displayed, the patient can reset and repeat the procedure until the patient achieves a suitable score. This can be accomplished by reinstalling and then removing closure cap 190. Alternatively, this can more easily be accomplished by merely depressing projection 416 twice, once to simulate insertion of closure of cap 190 and the second time to simulate removal of closure cap 190, thereby effectively resetting the device.

It will be appreciated that various modifications can be made to the present invention. For example, training device 310 can be disabled if the patient breathes into mouthpiece 370. In such case, training device 310 can be programmed to reactivate following a ten second interval.

Having described specific embodiments of the invention with reference to the accompanying drawings, it will be appreciated that the present invention is not limited to those precise embodiments and that various changes and modifications can be effected therein by one of ordinary skill in the art without departing from the scope or spirit of the invention as defined by the appended claims.

What is claimed is:

1. A medication inhaler training device, comprising:
a medication inhaler simulator including:
a housing having a bore extending therethrough, and
a mouthpiece connected with said housing and being in open communication with said bore; and
a control circuit for measuring pressure drop at a position below said mouthpiece, said control circuit including:
a pressure transducer for producing an output signal corresponding to said pressure drop at said position,
a display for providing an indication of acceptable inhalation, and
a microprocessor connected with said display for controlling said display in response to said output signal from said pressure transducer and elapsed time.

2. The training device according to claim 1,
wherein said medication inhaler simulator further includes an opening in a wall of said housing; and
further including a conduit having one end connected with said wall of said housing at said opening and an opposite end connected with said pressure transducer, wherein said control circuit measures said pressure drop at said opening.

3. The training device according to claim 2, wherein said housing includes a restriction arrangement for restricting air passage through a portion of said bore to provide adjustment of a pressure drop through said bore.

4. The training device according to claim 3, wherein said opening in said housing is positioned between said restriction arrangement and said mouthpiece.

5. The training device according to claim 1, wherein said mouthpiece is removably mounted on said housing.

6. The training device according to claim 5, wherein said mouthpiece is threadedly mounted on said housing.

7. The training device according to claim 1, wherein said housing includes a closed end covered by said mouthpiece, said closed end including:
a pressure sensing opening positioned above the pressure transducer, and
at least one air flow opening fluidly connected with an outside of said housing.

8. The training device according to claim 7, wherein said housing includes at least one recessed wall section positioned below said at least one air flow opening.

9. The training device according to claim 7, wherein said recessed wall section includes a curvature that reduces in depth in a radial dimension at an intermediate portion thereof to create an air flow restriction with said mouthpiece so as to create a venturi effect.

10. The training device according to claim 1, wherein said control circuit includes an analog-to-digital converter connected between said pressure transducer and said microprocessor for digitizing said output signal prior to supply thereof to said microprocessor.

11. The training device according to claim 1, wherein said control circuit includes a start switch for closing a power circuit in order to supply power to said microprocessor from a power supply.

12. The training device according to claim 11, wherein said control circuit further includes a timer circuit for opening said power circuit after a predetermined amount of time.

13. The training device according to claim 11, wherein said control circuit further includes a voltage regulator connected with said power supply through said power circuit, and which supplies a predetermined DC voltage to said microprocessor in response to power supplied by said power supply.

14. The training device according to claim 11, wherein said control circuit further includes a voltage detector connected with said microprocessor, for detecting voltage supplied by said power supply, and for sending a signal to said microprocessor when said voltage is below a predetermined value in order to prevent operation of said microprocessor.

15. The training device according to claim 1, wherein said display includes at least one lighting device which is selectively caused to be illuminated by said microprocessor for providing an indication of both inhalation rapidity and inhalation flow rate peak.

16. The training device according to claim 15, wherein said at least one lighting device include a plurality of light emitting diodes, and further comprising drivers connected between said light emitting diodes and said microprocessor.

17. The training device according to claim 15, wherein said at least one lighting device is arranged to display said inhalation rapidity and said inhalation flow rate peak in a bar graph form.

18. The training device according to claim 15, wherein said at least one lighting device includes a liquid crystal display.

19. The training device according to claim 1, wherein said display includes a lighting device which is selectively caused to be illuminated by said microprocessor to provide a single display which indicates whether inhalation is acceptable.

20. The training device according to claim 19, wherein said single display is a function of values of both inhalation rapidity and inhalation flow rate peak.

21. The training device according to claim 20, wherein said single display includes a plurality of bars which are selectively illuminated in dependence upon a single value calculated from the values of both inhalation rapidity and inhalation flow rate peak.

22. The training device according to claim 21, wherein different ones of said display bars are illuminated with different colors.

23. The training device according to claim 21, wherein there are a plurality n of said bars which are selectively illuminated in dependence upon said single value calculated as follows:
a) if the values of both inhalation rapidity and inhalation flow rate peak are below a threshold value m which is less than n, the lower value of inhalation rapidity and inhalation flow rate peak is displayed as the single value, providing that the flow rate at a predetermined time from the start of inhalation is above a predetermined flow rate, to indicate an unsuccessful inhalation, wherein at least one said bar corresponding to said single value is illuminated,
b) if the values of both inhalation rapidity and inhalation flow rate peak are at least as high as the threshold value, an average truncated value of the values of both inhalation rapidity and inhalation flow rate peak is displayed as the single value, providing that the flow rate at the predetermined time from the start of inhalation is above the predetermined flow rate, to indicate a successful inhalation, wherein a number of said bars corresponding to said single value are illuminated, and
c) if the flow rate at the predetermined time from the start of inhalation is below the predetermined flow rate, to indicate an unsuccessful inhalation, wherein at least one said bar corresponding to said single value is illuminated.

24. The training device according to claim 23, wherein m equals five and n equals ten.

25. The training device according to claim 1, wherein said control circuit is mounted within said housing.

26. The training device according to claim 25, wherein said housing includes a viewing opening, and said display is positioned to be viewed through said viewing opening.

27. The training device according to claim 1, wherein said mouthpiece includes a one-way valve to inhibit contamination of said housing if a patient exhales into the mouthpiece.

28. The training device according to claim 27, wherein said one-way valve includes a retainer having openings mounted in said mouthpiece, and a flexible valve flap mounted on said retainer which moves away from said retainer to permit air flow through said retainer and said mouthpiece during inhalation and which blocks said openings in said retainer during exhale by the patient through the mouthpiece.

29. The training device according to claim 1, wherein:
said control circuit includes a start switch for closing a power circuit in order to supply power to said microprocessor from a power supply; and
the training device further comprises:
a closure cap removably positioned over said mouthpiece, and
an actuation assembly in said housing for actuating said start switch upon removal of said closure cap.

30. The training device according to claim 29, wherein said actuation assembly includes:
a ring mounted for rotation in said housing, said ring including an actuating projection for actuating said start switch upon rotation of said ring, and a first engaging portion; and
a second engaging portion mounted to said closure cap for engagement with said first engaging portion to rotate said ring.

31. The training device according to claim 30, wherein:
said housing includes external threads, and said closure cap includes internal threads for threadedly positioning said closure cap on said housing in covering relation to said mouthpiece;
said first engaging portion extends outwardly of said housing through at least one opening in a wall of said housing; and
said second engaging portion is formed on an inner surface of said closure cap so as to rotate said ring in a first direction upon threaded removal of said closure cap and to rotate said ring in a second direction upon threaded insertion of said closure cap on said housing.

32. A method of assessing a patient's inhalation process with respect to a medication inhaler, comprising the steps of:
inhaling through a mouthpiece of an inhaler simulator having a housing with a bore extending therethrough, with the mouthpiece connected with said housing and being in open communication with said bore;
measuring a pressure drop at a position below said mouthpiece during inhalation through said mouthpiece, and providing an output signal in response thereto;
determining inhalation rapidity in response to said output signal and an elapsed time;
determining an inhalation flow rate peak in response to said output signal; and
providing a display in response to both said inhalation rapidity and said inhalation flow rate peak.

33. The method according to claim 32, wherein said steps of determining inhalation rapidity and determining inhalation flow rate peak are performed in a microprocessor, and further comprising the step of digitizing said output signal prior to supply thereof to said microprocessor.

34. The method according to claim 33, further comprising the step of shutting off power to said microprocessor after a predetermined amount of time.

35. The method according to claim 32, wherein said step of determining inhalation rapidity includes the steps of:
reading the output signal;
determining a starting time when said output signal reaches a predetermined initial value;
determining an ending time when said output signal reaches a predetermined ending value;
determining a time period between said initial value and said ending value; and
determining said inhalation rapidity from a difference between said ending value and said initial value over said time period.

36. The method according to claim 32, wherein said step of displaying includes the step of illuminating at least one lighting device to display said inhalation rapidity and said inhalation flow rate peak in a bar graph form.

37. The method according to claim 32, wherein said step of displaying includes the step of illuminating at least one lighting device to display an indication of whether inhalation is acceptable.

38. The method according to claim 37, wherein the step of displaying includes the step of selectively illuminating a plurality of bars dependence upon a single value calculated from the values of both inhalation rapidity and inhalation flow rate peak.

39. The method according to claim 38, wherein said step of selectively illuminating includes the step of illuminating the different ones of said display bars with different colors.

40. The method according to claim 37, wherein there are a plurality n of said bars, and the step of displaying includes the steps of:
a) illuminating at least one said bar corresponding to a single lower value of inhalation rapidity and inhalation flow rate peak if the values of both inhalation rapidity and inhalation flow rate peak are below a threshold value m which is less than n, providing that the flow rate at a predetermined time from the start of inhalation is above a predetermined flow rate, to indicate an unsuccessful inhalation,
b) illuminating a number of said bars corresponding to an average truncated value of the values of both inhalation rapidity and inhalation flow rate peak if the values of both inhalation rapidity and inhalation flow rate peak are at least as high as the threshold value, providing that the flow rate at the predetermined time from the start of inhalation is above the predetermined flow rate, to indicate a successful inhalation, and
c) illuminating at least one said bar corresponding to a single value if the flow rate at the predetermined time from the start of inhalation is below the predetermined flow rate, to indicate an unsuccessful inhalation.

41. The method according to claim 40, wherein m equals five and n equals ten.
